(19) United States
(12) Patent Application Publication
(10) Pub. No.: US 2012/0253279 A1
(43) Pub. Date: Oct. 4, 2012

(54) CATHETER INTRODUCTION

(76) Inventor: Patrick Stuart Thorp, Nantwich (GB)

(21) Appl. No.: 13/370,623

(22) Filed: Feb. 10, 2012

(30) Foreign Application Priority Data
Feb. 11, 2011 (GB) 1102424.7

(51) Int. Cl. A61M 29/02; A61B 17/34; A61M 25/08

(52) U.S. Cl. 604/96.01; 604/528; 604/170.02; 604/164.05; 604/164.11

(57) ABSTRACT

A method of introducing a catheter 38 into an umbilical artery 14. The artery 14 is dilated with an introducer 22. A cannula 30 is introduced into the end of the artery 14, and a catheter is introduced through the cannula 30, with the distal end of the catheter 38 not engaging with the cut end of the artery 14.

### FIELD OF THE INVENTION

This invention concerns a method of introducing a catheter into an umbilical artery.

### BACKGROUND TO THE INVENTION

Umbilical artery catheters are frequently used for blood pressure monitoring, and are also used for blood sampling. Severe adverse incidents with such catheters are uncommon, but in trials aortic thrombi have been detected in 30% of cases. Also at a level of around 30% minor perfusion problems in the feet have been encountered, often resolving with contralateral limb warming, at times requiring catheter removal, and occasionally causing loss of digits. The cause is currently thought to be arterial ‘spasm’.

Adverse incidents may occur due to malpositioning of the catheter, and correct positioning is considered to be lower thoracic or lower lumbar aorta. The tip is intended to lie within the aorta. Other adverse incidents may be due to the technique of introduction. One such feature may be the tip of the catheter being contaminated by coming into contact with the cut end of the umbilical stump.

Conventionally the catheter is manually inserted after dilating the cut end of the artery. A no touch technique may be used, but the tip of the catheter becomes exposed to the cut end of the artery, and also at times to the surrounding Wharton's jelly, either directly and/or indirectly by the gloved hand or instruments.

The cut end of the artery contains procoagulants. Wharton's jelly contains substances that cause red cell agglutination. These substances are sufficient cause in the pathogenesis of thrombosis, or minor red cell agglutinations, both involved in the production of arterial emboli, which are a more logical cause of the pattern of symptoms.

### BRIEF DESCRIPTION OF VARIOUS EMBODIMENTS OF THE INVENTION

According to the present invention there is provided a method of introducing a catheter into an umbilical artery, the method comprising providing a barrier between the distal end of the catheter and the cut end of the artery and surrounding Wharton's jelly, as the catheter is initially introduced into the artery.

The barrier may be removed subsequent to initial introduction of the catheter into the artery.

The artery may be dilated prior to initial introduction of the catheter thereinto, and the dilation may be carried out by any of forceps, an introducer with a rounded end, or an obturator with a rounded end.

In a first embodiment a cannula is provided which lines the inner walls of the proximal end of the artery, and the catheter is slidably movable through the cannula.

The cannula may have a proximal flange which engages against the umbilicus around the end of the artery.

The cannula may be of a sufficient length to extend into a patient's abdomen when located extending into the umbilical artery.

The cannula may be formed such that it can be removed from around the catheter following location of the catheter in the artery. A line of weakness may be provided on the cannula to allow it to be broken open to permit removal from around the catheter.

One or more co-operable formations may be provided on the cannula which enable selective opening and closing thereof, with the catheter removable from the cannula when open.

The cannula may be formed of a plastics material, or alternatively of metal.

The cannula may be selectively locatable on a locating tool, which tool can be used to locate a cannula mounted thereon in the proximal end of the artery, with the cannula removable from the tool once the cannula is in place in the artery.

The locating tool may include an obturator on which the cannula can be located. The locating tool may be made of a flexible material.

The obturator may have a rounded distal end. The obturator may taper towards the distal end thereof. The obturator may be longer than the cannula such that when a cannula is located thereon, the distal end of the obturator extends beyond the cannula.

The locating tool may include a step at the proximal end of the obturator, such that the proximal end of the cannula engages against the step.

The locating tool may also include an introducer engageable in an artery to dilate same. The introducer may have a rounded distal end. The introducer may have a greater diameter than the obturator. The introducer may be provided on an opposite end of the locating tool to the obturator.

In a second embodiment of the invention, a film is provided over the distal end of the catheter prior to introduction into the artery, to close the distal end of the catheter, and the film may be broken following introduction of the catheter into the artery to open the distal end of the catheter.

The film may be made of plastics material. The film may be arranged such that the cannula can pass therethrough once the film has been broken.

### BRIEF DESCRIPTION OF THE DRAWINGS

Embodiments of the present invention will now be described by way of example only and with reference to the accompanying drawings, in which:

FIG. 1 is a diagrammatic perspective view of an umbilicus;

FIG. 2 is a sectional side view of apparatus usable in a method according to the invention;

FIG. 3 is a side view of part of the apparatus of FIG. 2;

FIG. 4 is a similar view to FIG. 3 of an alternative apparatus;

FIG. 5 is a diagrammatic cross sectional view through an umbilicus showing the method being carried out;

FIG. 6 is a diagrammatic side view of further apparatus usable with the invention;

FIG. 7 is a similar view to FIG. 2 of a further alternative apparatus.

### DETAILED DESCRIPTION OF VARIOUS EMBODIMENTS OF THE INVENTION

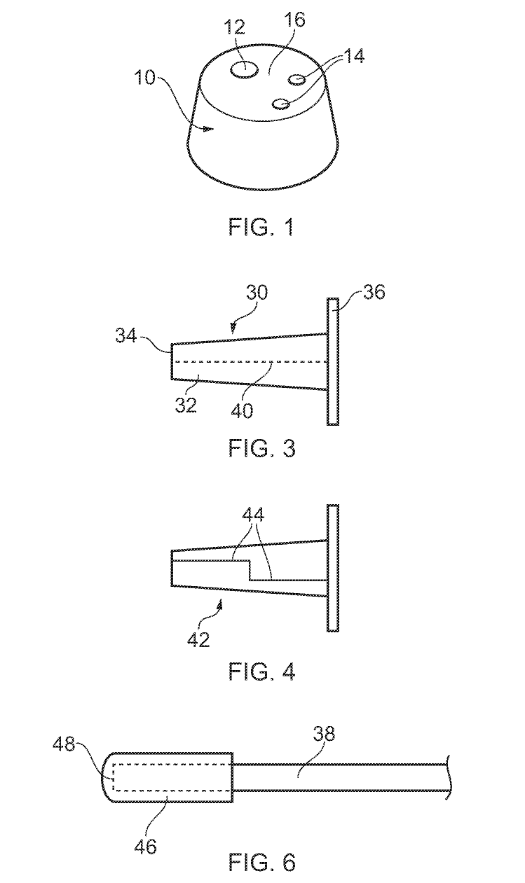

FIG. 1 shows an umbilicus 10, with an umbilical vein 12 and two umbilical arteries 14. The umbilicus 10 is orientated with the cephalad (head) to left. The vein 12 and arteries 14 are held in Wharton's jelly 16. As indicated above it is often required to introduce a catheter into a one of the arteries 14 for blood pressure monitoring and/or for blood sampling.

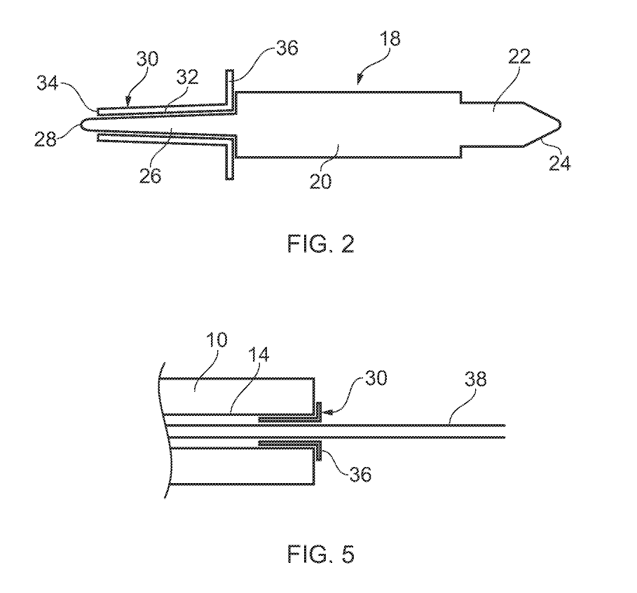

FIG. 2 illustrates apparatus usable in the method of inserting a catheter in a one of the veins 14. FIG. 2 shows a locating tool 18 with a central cylindrical body 20. At one end of the body 20 an introducer 22 is provided. The introducer 22 is of a smaller diameter than the body 20 and has a rounded end 24. At the opposite end of the body 20 an obturator 26 is provided which is of a smaller diameter than the introducer 12. The obturator 26 tapers gently to a rounded end 28.

A cannula 30 shown in FIG. 2 located on the obturator 26. The cannula 30 is of a size to slidingly fit on the obturator 26, with the rounded end 28 extending beyond the cannula 30. The cannula 30 has a hollow cylindrical body 32 which tapers gently to a distal end 34. A flange 36 is provided at the proximal end of the cannula 30, and the flange 36 is engageable against the body 20.

To locate a catheter in a one of the arteries 14, the respective artery 14 will be dilated. This may require insertion of the introducer 22, forceps, and/or introduction of the obturator 26. The obturator 26 with the cannula 30 located thereon as shown in FIG. 2, is introduced into the proximal end of the respective artery 14. The locating tool 18 is slid out of the cannula 30, leaving the cannula 30 lining the proximal end of the artery 14, as shown in FIG. 5.

A catheter 38 can then be slowly introduced through the cannula 30, with the distal end of the catheter 38 not engaging with the cut end of the artery 14 and hence the procoagulants therein. Also the distal end of the catheter 38 does not engage with the Wharton's jelly 16 and hence the substances that can cause red cell agglutination. Once the catheter 38 has passed through the cannula 30, and perhaps to a required position, the cannula 30 is slid out of the artery 14 along the catheter.

FIG. 3 shows the cannula 30 with a line of weakness 40 extending along the cylindrical body 32. Once the cannula 30 has been removed from the artery 14, the cannula 30 can be broken along the line 40 and removed from off the catheter 38. In this instance the cannula 30 is made of a plastics material and is sufficiently flexible to be readily pulled off the catheter 38.

FIG. 4 shows an alternative cannula 42, in this instance made of metal. The cannula 42 is similar to the cannula 30, but rather than a line of weakness, cooperable formations 44 are provided to permit selective opening of the cylindrical body to enable removal from around the catheter 38.

FIG. 6 diagrammatically illustrates a further method according to the invention. In this instance a plastics material film 46 is provided around the distal end 48 of the catheter 38, with the film 46 closing the distal end 48. Once the catheter 38 has been inserted beyond the proximal end of the artery 14, the end of the film 46 can be broken to provide access to the distal end 48. It may be that the end of the film 46 is ruptured, but the film left in position on the end of the catheter 38. Alternatively the film 46 may be held in position and the catheter 38 slid therethrough, such that the distal end 48 becomes spaced from the film 46.

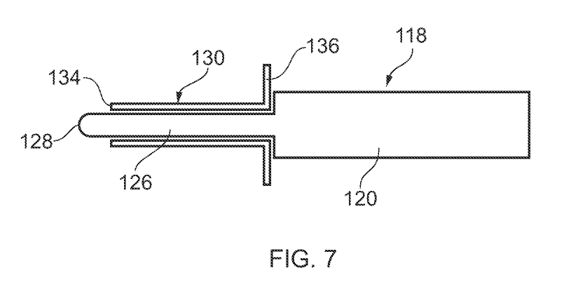

FIG. 7 is a similar view to FIG. 2 showing an alternative locating tool 118. The tool 18 again has a cylindrical body 120, but no introducer is provided. The body 120 acts as a handle for the tool 118. In this instance the obturator 126 does not taper, and has a more rounded distal end 128.

A further cannula 130 is shown located on the obturator 26. The cannula 130 is of a size to slidingly fit on the obturator 126, and in this instance did not taper towards its distal end 134. Otherwise the cannula 130 has a similar formation to that shown in FIG. 2, with a flange 136.

It is to be realised that the locating tool and/or cannula can take a number of different forms, and may include any combination of the above described features. For instance a tapering obturator with a rounded end may be used. The locating tool may be made of a flexible material.

There are thus described methods, and apparatus usable with such methods to enable catheters to be inserted into an umbilical artery without the end of the catheter coming into contact with the end of the artery and also the surrounding Wharton's jelly. Accordingly the adverse incidents outlined above can be avoided.

It is to be realised that various other modifications may be made without departing from the scope of the invention. For instance a cannula with a different form could be used. Other methods may be used for initially providing a barrier between the distal end of the catheter.

Whilst endeavouring in the foregoing specification to draw attention to those features of the invention believed to be of particular importance it should be understood that the Applicant claims protection in respect of any patentable feature or combination of features hereinbefore referred to and/or shown in the drawings whether or not particular emphasis has been placed thereon.

1. A method of introducing a catheter into an umbilical artery, the method comprising providing a barrier between a distal end of the catheter and a cut end of the artery and surrounding Wharton's jelly, as the catheter is initially introduced into the artery.

2. A method according to claim 1, wherein the barrier is removed subsequent to initial introduction of the catheter into the artery.

3. A method according to claim 1, wherein the umbilical is dilated prior to initial introduction of the catheter thereinto.

4. A method according to claim 3, wherein dilation of the umbilical artery is carried out by any of forceps, an introducer with a rounded end, or an obturator with a rounded end.

5. A method according to claim 1, wherein a cannula is provided which lines the inner walls of the end of the artery, and the catheter is slidably movable through the cannula.

6. A method according to claim 5, wherein the cannula has a proximal flange which engages against the umbilicus around the end of the artery.

7. A method according to claim 5, wherein the cannula is of a sufficient length to extend into a patient's abdomen when located extending into the umbilical artery.

8. A method according to claim 5, wherein the cannula is formed such that it can be removed from around the catheter following location of the catheter in the artery.

9. A method according to claim 8, wherein a line of weakness is provided on the cannula to allow it to be broken open to permit removal from around the catheter.

10. A method according to claim 8, wherein one or more co-operable formations is provided on the cannula which enable selective opening and closing thereof, with the cannula removable from around the catheter when open.

11. A method according to claim 5, wherein a locating tool is provided, and the cannula is selectively locatable on the locating tool, which tool can be used to locate a cannula mounted thereon in the end of the artery, with the cannula removable from the tool once the cannula is in place in the artery.

12. A method according to claim 11, wherein the locating tool includes an obturator on which the cannula can be located.

13. A method according to claim 12, wherein the obturator is longer than the cannula such that when a cannula is located thereon, a distal end of the obturator extends beyond the cannula.

14. A method according to claim 12, wherein the locating tool includes a step at a proximal end of the obturator, such that the proximal end of the cannula engages against the step.

15. A method according to claim 11, wherein the locating tool includes an introducer engageable in an artery to dilate same.

16. A method according to claim 15, wherein the locating tool includes an obturator on which the cannula can be located, and the introducer has a greater diameter than the obturator.

17. A method according to claim 16, wherein the introducer is provided on an opposite end of the locating tool to the obturator.

18. A method of introducing a catheter into an umbilical artery, the method comprising providing a barrier between a distal end of the catheter and a cut end of the artery and surrounding Wharton's jelly, as the catheter is initially introduced into the artery, the barrier being provided by a film provided over the distal end of the catheter prior to introduction into the artery to close the distal end of the catheter, the film being breakable following introduction of the catheter into the artery to open the distal end of the catheter.

19. A method according to claim 18, wherein the film is made of plastics material.

20. A method according to claim 18, wherein the film is arranged such that the cannula can pass therethrough once the film has been broken.
